(19) United States
(12) Patent Application Publication
(10) Pub. No.: US 2007/0003595 A1
(43) Pub. Date: Jan. 4, 2007

(54) Three dimensional micro-environments and methods of making and using same

(76) Inventors: Shaopeng Wang, Stillwater, OK (US); Nicholas Kotov, Stillwater, OK (US); Jun Zhang, Xincheng District Hohhot (CN)

Correspondence Address: DUNLAP, CODDING & ROGERS P.C., PO BOX 16370, OKLAHOMA CITY, OK, 73113, US

(21) Appl. No.: 11/407,002

(22) Filed: Apr. 19, 2006

Related U.S. Application Data
(60) Provisional application No. 60/672,762, filed on Apr. 19, 2005.

(51) Int. Cl. C12M 3/00; A61K 35/12; A61F 2/02

(52) U.S. Cl. 424/426; 435/289.1

(57) ABSTRACT

The presently claimed and disclosed invention relates, in general, to three dimensional micro-environments and, in particular, to three dimensional (“3D”) micro-environments found in inverted-opal scaffolds made from hydrogel therein for controlled release of nutrients. Specifically, the scaffolds have exceptionally ordered, three-dimensional organization that provides excellent porosity, permeability, and transportation properties can that are especially well suited for use as a nutrient carrier in the emerging technologies of drug delivery and cell culture. Methods for incorporation of or to control the release of nutrients and other substances from such scaffold materials are also herein disclosed and claimed.

### CROSS REFERENCE TO RELATED CASES

The present invention claims benefit under 37 C.F.R. 119(e) to provisional patent application U.S. Ser. No. 60/672,762, filed Apr. 19, 2006 and entitled “THREE DIMENSIONAL MICRO-ENVIRONMENT AND METHODS OF MAKING AND USING SAME”, the entire contents of which are hereby incorporated by reference in their entirety as set forth explicitly herein.

### BACKGROUND OF THE INVENTION

1. Field of the Invention

The presently claimed and disclosed invention relates, in general, to three dimensional micro-environments and, in particular, to three dimensional (“3D”) micro-environments incorporating inverted-opal scaffolds made from hydrogel therein for controlled release nutrients. Specifically, the scaffolds have exceptionally ordered, three-dimensional organization that provides excellent porosity, permeability, and transportation properties that are especially well suited for use as a nutrient carrier in the emerging technologies of drug delivery and cell culture. Methods to control the release of nutrients and other substances from such scaffold materials are also herein disclosed and claimed.

2. Background of the Invention

Controlled release of bioactive molecules (e.g., protein, drug and DNA etc.) from a carrier material into a specified micro-environment at a designed rate is of significance for biological uses such as drug delivery and cell culture. Drug delivery requires that the bio-degradable carrier materials are capable of: (1) alleviating the possible toxicity; (2) promoting the therapeutic level; (3) improving the availability of drugs; and (4) directly interacting with the desired targets. The activation of these functionalities with carrier materials is determined primarily by their ability to release the molecules of interest in a controlled manner.

Cell culture processes require that the carrier materials not only be a biocompatible matrix or scaffold for cell adhesion but that they can also act as a reservoir to store nutrient, growth factors and differentiation factors and thereafter release these nutrients in a controlled manner that favors cell growth and differentiation. The controlled release of nutrients from carrier materials is critically important to cell growth and differentiation. If the release rate is controlled at a designed rate, the cell growth and differentiation is greatly promoted. Therefore, the development of a controlled release system is of great scientific and technological importance.

Recently, there has been significant interest in the development of “smart” carrier materials for drug delivery and cell culture methodologies. The ideal carrier materials for drug delivery and cell culture are degradable, porous and highly permeable, able to maintain a desired microenvironment, and capable of precisely controlled release of bioactive species such as nutrients, growth factors and differentiation factors.

Numerous materials (e.g., inorganic ceramics, organic architectures and polymer networks) have been developed as scaffold or matrix materials for storing the protein, DNA and peptide for drug delivery and cell culture. These materials are mostly in the forms of a mixture, gel or film which are difficult to fabricate such that they are capable of releasing bioactive molecules in a controlled manner. The excellent mechanical properties of inorganic ceramics make them useful for mimicking bone components; however, the rigid inorganic structure has very limited storing capability. Inorganic ceramics are, therefore, poor candidates for the controlled release of carrier materials.

Organic and polymer matrices are also thought to be promising candidates for controlled release carrier materials due to their network structure which is capable of holding and releasing biomolecules under certain conditions. The network structures of these matrices are, unfortunately, such that the small pores of these materials limit the transportation of larger protein molecules.

Hydrogel materials used as a controlled release carrier have been widely investigated (e.g., see U.S. Pat. No. 4,749,576). Hydrogels are superabsorbent natural or synthetic polymers. By properly selecting the hydrogel macromers, hydrogels can be produced as a membrane with a range of permeability, pore size and degradation rates suitable for applications in drug delivery or cell culture. Hydrogels, therefore, have the possibility of carrying a species (i.e., molecules of interest, protein molecules, DNA molecules, etc.) that could thereafter be released in a controlled manner. Due to the lack of ordered pore size and poorly controllable architectures of hydrogel membranes, however, they are unable to release the carried species in a controlled manner and are, therefore, ill-suited for use as a controlled release carrier. To this point, conventional hydrogel materials (such as collagen) formed into a film or gel are also not suitable as a controlled release carrier for uses in drug delivery or cell culture. A well controlled 3D ordered structure that can be utilized both as a scaffold and as a carrier is thus needed in the field.

The importance of 3D organization of cells has been demonstrated on a variety of cellular cultures on different 3D scaffolds. Despite the variety of the geometries and materials used for 3D supports, the consensus is that tissue functions are replicated better in the 3D environment rather than in standard 2D systems. In part for that reason, many 3D supports have been considered for potential use as implants for tissue repair, which encompasses both hard and soft tissues. In this respect, the ability of this matrix to release bioactive species (proteins, drugs and DNAs etc.) at a designed rate is of great significance for tissue engineering. This functionality can recruit stem and other types of cells, reduce inflammation, improve compatibility with surrounding tissue, stimulate development of desirable type of tissues, and perform other functions critical for the healing process.

An inverted-opal material has such a 3D structure and is disclosed and claimed herein as a novel controlled release carrier. The topography of the inverted-opal consists of spherical cavities interconnected by a network of channels that favors the transportation, storage and delivery of bioactive species. Inverted colloidal crystals (ICC) are inverted-opal structures with a highly ordered (crystalline), 3D structure; inverted-opals may have a less ordered structure or more defects. ICCs can be made by using a colloidal crystal as a template, according to a recently published technique (i.e., see U.S. patent application Ser. No. 10/460,059 “3D Tissue Constructs on the basis of colloidal crystals surface modified by sequential layering”; Y. Zhang, S. Wang, M. Eghtedari, M. Motamedi, N. A. Kotov, Inverted Colloidal Crystal Hydrogel Matrices as Three-Dimensional (3D) Cell Scaffolds, Advanced Functional Materials, 2005, 15, 725-731; N. A. Kotov, Y. Liu, S. Wang, C. Cumming, M. Eghtedari, G. Vargas, M. Motamedi, J. Nichols, J. Cortiella, Inverted Colloidal Crystals as 3D Cell Scaffolds, Langmuir, 2004, V20, No.19, 7887-7892; Y. Liu, S. Wang, N. A. Kotov, A Floating Self-Assembly Route to Colloidal Crystal Templates for 3D Cell Scaffolds, Chem. Mater., 2005 17(20); 4918-4924; D. J. Irvine, A. Stachowiak, S. Jain, Engineered Biomaterials for Control of Immune Cell Functions, Materials Science Forum, 2003, 426, 3213-3218). The advantages of ICC scaffolds include exceptionally high 3D order and reproducibility, efficient nutrient transport, functional flexibility and large surface area for cell adhesion. By means of computer modeling, it has also been shown that the ICC topography favors the facile transport of nutrients (i.e., see S. Shanbhag, S. Wang, N. A. Kotov, Cell Distribution Profiles in Three-Dimensional Scaffolds with Inverted Colloidal Crystal Geometry: Modeling and Experimental Investigations, Small, 2005, V 1, No. 12, 1208-1214; S. Shanbhag, J. W. Lee, N. A. Kotov, Diffusion in three-dimensionally ordered scaffolds with inverted, colloidal crystal geometry, Biomaterials 26 (2005) 5581-5585) The development of 3D inverted-opal and ICC structures with hydrogel materials meets the above-mentioned requirements of a controlled release carrier of molecules of interest (i.e., nutrients, growth and differentiation factors for cell culture and drug delivery) that are necessary for a useful and novel 3D micro-environment.

### SUMMARY OF THE INVENTION

Substances such as nutrients, growth and differentiation factors that are needed by cells for growth and differentiation can be incorporated into inverted-opal scaffolds made from hydrogel material. Such substances can be incorporated into the scaffold during the fabrication process, or they may be incorporated post fabrication though diffusion. The release kinetics of the substances can be controlled in several ways: 1) by controlling incorporation concentration, 2) by controlling hydrogel permeability (i.e. controlling polymer cross-linking rate) and 3) by using a layer-by-layer (LBL) coating on the scaffold. In this manner, the substance concentrations over time in the three dimensional micro-environments found inside the scaffolds can be precisely controlled for optimal cell growth and differentiation.

According to the presently disclosed and claimed invention, there are provided methods that are capable of controlling nutrient release through 3D ordered hydrogel scaffolds for drug delivery or cell growth and differentiation uses. For example, one embodiment provides a method for the controlled release of one or more constituent compounds. This method, more particularly, includes the steps of (1) providing an inverted-opal scaffold, wherein the inverted-opal scaffold comprises a hydrogel material; and (2) incorporating one or more constituent compounds in the inverted-opal hydrogel scaffold, wherein the one or more constituent compounds incorporated in the inverted-opal hydrogel scaffold are capable of being released from the inverted-opal hydrogel scaffold in a controlled manner.

In the method outlined above, the constituent compounds may, in one embodiment, be selected from the group consisting of biological materials, chemicals, polymers, naturally occurring compounds, synthetic compounds, and combinations thereof, while in an alternative embodiment the constituent compounds may be selected from the group consisting of nutrients, growth factors, differentiation factors, protein, DNA, peptides, cytokines, chemokines, drugs, and combinations thereof. The incorporation of the one or more constituent compounds into the inverted-opal hydrogel scaffold may occur by mixing the one or more constituent compounds with the precursor materials used to produce the inverted-opal hydrogel scaffold or the constituent compounds may be incorporated by diffusing the constituent compounds into the inverted-opal hydrogel scaffold after its production. Alternatively, the constituent compounds may be incorporated into the inverted-opal hydrogel scaffold by active transport.

It is also to be understood that the presently disclosed and claimed inventions also include the ability to control the release rate of the one or more constituent compounds out of the inverted-opal hydrogel scaffold. For example, the kinetics of release of the one or more constituent compounds may be controlled by the concentration of constituent compounds incorporated into the inverted-opal hydrogel scaffold or the release may be controlled by controlling one or more properties of the inverted-opal hydrogel scaffold. Such properties may include those that are selected from the group consisting of permeability, pore size, channel size, degree of cross-linking, elasticity, hardness, and combinations thereof.

In an alternate embodiment, the kinetics of the release of the one or more constituent compounds may be controlled by including a coating onto the surface of the inverted-opal hydrogel scaffold and such a coating may, in one such embodiment, be produced according to a layer-by-layer methodology. Also, it is contemplated that the kinetics of the release of the one or more constituent compounds may be controlled by the number of layers or materials used in the coating.

In yet another embodiment, the kinetics of the incorporation or the controlled release of the one or more constituent compounds into or out of the inverted-opal hydrogel scaffold may be controlled by one or more environmental parameters selected from the group consisting of salt concentration, temperature, pH, and combinations thereof. Finally, it is contemplated that the kinetics of the release of the one or more constituent compounds may be controlled by the degradation of the hydrogel material in the inverted-opal hydrogel scaffold.

In at least one embodiment of the method outlined above, the inverted-opal hydrogel scaffold further includes a secondary material operably associated with the hydrogel itself, or with the totality of the inverted-opal hydrogel scaffold so as to form a composite scaffold within or with the inverted-opal hydrogel scaffold. Also, it is contemplated that the inverted-opal hydrogel scaffold may have an inverted colloidal crystal structure.

The presently disclosed and claimed inventions also include a method of providing a 3D microenvironment for cell growth. This method includes the steps of (1) providing an inverted-opal hydrogel scaffold having a three-dimensional structure; (2) incorporating one or more constituent compounds into the inverted-opal hydrogel scaffold; and (3) controlling the release of the one or more constituent compounds into a medium that permeates through the inverted-opal hydrogel scaffold, to thereby provide a predetermined concentration of the one or more constituent compounds for cell growth. Additionally, all of the embodiments discussed for the first method outlined hereinabove are equally applicable and useful for use with this method.

In one alternate embodiment to this method, the predetermined concentration of the one or more constituent compounds for cell growth is an optimal concentration of the one or more constituent compounds capable of optimizing cell growth.

### BRIEF DESCRIPTION OF THE SEVERAL VIEWS OF THE DRAWINGS

FIG. A are photographs showing batch produced ICC hydrogel scaffolds. Left: cylindrical master hydrogel ICC scaffolds; Center: individual ICC scaffolds obtained by cutting the master scaffolds; Right: Scanning electron microscope (SEM) image of the structure of the as produced ICC scaffold.

FIG. 1 is a graphical representation showing the fluorescence spectra of Inhibitor from Tripsin Soybean, Alexa Fluor 488 labeled (SBTI) PBS solution (40 ng/ml initial concentration) measured at different time intervals after the hydrogel scaffolds were introduced. The fluorescence intensity decreases with the scaffold stand time in SBTI solution, indicating the diffusion of SBTI into the scaffolds, (a) S1 and (b) S2.

FIG. 2 is a graphical representation showing the kinetics of SBTI diffusion into hydrogel scaffolds, demonstrated by peak fluorescence intensity at 516 nm. Both curves show exponential decay, and the scaffolds are saturated with SBTI around 3 days.

FIG. 3 is a graphical representation showing the exponential decay of fluorescence from a fluorescence-labeled SBTI PBS solution.

FIG. 4 is a graphical representation showing the corrected kinetics of SBTI diffusion into hydrogel scaffolds. Data show that the diffusion of SBTI into hydrogel scaffolds occurs mostly within the first four hours, as shown by the sharp decrease of fluorescence intensity after such a time period.

FIG. 5 is a graphical representation showing the corrected kinetics of SBTI release out of hydrogel scaffolds. The data indicates that the release of SBTI out of hydrogel scaffolds can be up to 648 hours (27 days).

FIG. 6 is a graphical representation showing the kinetics of SBTI release out of the LBL coated Na₂SiO₃ scaffolds. Curves of A, B, C, D, and E are corresponding to the fluorescence variations of Na₂SiO₃scaffold-(PDDA-SBTI)2, Na₂SiO₃ scaffold-(PDDA-SBTI)4, Na₂SiO₃ scaffold-(PDDA-SBTI)6, Na₂SiO₃ scaffold-(PDDA-SBTI)8-PDDA, and Na₂SiO₃ scaffold-(PDDA-SBTI)10.

FIG. 7 is a graphical representation showing the kinetics of SBTI release out of the LBL coated pHEMA hydrogel scaffolds. Curves of A, B, C, D, E and F are corresponding to the fluorescence variations of pHEMA scaffold (PDDA-SBTI)1, pHEMA scaffold (PDDA-SBTI)2, PHEMA scaffold (PDDA-SBTI)4, PHEMA scaffold (PDDA-SBTI)6, pHEMA scaffold (PDDA-SBTI)8-PDDA, and pHEMA scaffold (PDDA-SBTI)10.

FIG. 8 is a graphical representation showing the kinetics of SBTI diffusion into hydrogel scaffold. The scaffold absorbs 1.08 ng per mm³ within 4 hours. 42% (1.08 of 2.56 ng/mm³) of SBTI (40 ng/ml, 4 mL) was absorbed into the scaffold (V˜62.5 mm³) within 4 hours.

FIG. 9 is a graphical representation showing the kinetics of SBTI release out of hydrogel scaffold both with and without LBL produced coatings. For the one without LBL coating, 88% (0.95 ng of 1.08 ng/mm³) of previously absorbed nutrient in scaffold is gradually released within the time up to 27 days. For the scaffold with LBL coating, only 42% (0.42 of 1.08 ng/mm³) of absorbed nutrient in scaffold is released within 3 days. It is shown that with LBL coating the release rate of nutrient from hydrogel scaffolds can be lowered from 88% to 42%, and the release time can be shortened from 27 to 3 days.

FIG. 10 is a graphical representation showing the IL-3 release kinetics of PAAM scaffold, left: one day release, right: one week release.

FIG. 11 is a graphical representation showing the effects of hydrogel scaffold released IL-3 on TF-1 cell based assays.

FIG. 12 is a graphical representation showing the cumulative IL-3 released from different hydrogel and composite scaffolds

FIG. 13 is a graphical representation showing the cumulative IL-3 release from positively-charged hydrogel scaffold compared to other hydrogel scaffolds.

FIG. 14 is a graphical representation showing the effects on TF-1 cell based assays of positively-charged hydrogel scaffold compared to other hydrogel scaffolds, for various methods of IL-3 delivery (or no IL-3) which are illustrated above.

### DETAILED DESCRIPTION OF THE PRESENTLY DISCLOSED AND CLAIMED INVENTION

Before explaining at least one embodiment of the invention in detail, it is to be understood that the invention is not limited in its application to the details of construction, experiments, exemplary data, and/or the arrangement of the components set forth in the following description or illustrated in the drawings. The invention is capable of other embodiments or of being practiced or carried out in various ways. Also, it is to be understood that the phraseology and terminology employed herein is for purpose of description and should not be regarded as limiting.

### EXAMPLE 1

### Synthesis of Hydrogel Inverted-Opal Scaffold

One method to synthesize ICC scaffold is described in (Y. Zhang, S. Wang, N. A. Kotov, Inverted Colloidal Crystal Hydrogel Matrices as Three-Dimensional (3D) Cell Scaffolds, Advanced Functional Materials, 2005, V 15, 728-731) and may be briefly described as a process according to the following steps:

(1) Self-Assembly of Colloidal Crystals from Monodisperse Micron-Scale PMMA Spheres.

1 mL ethanol was added to 0.1 g of 104 μm monodisperse poly(methyl methacrylate) colloid (PMMA, from Microparticles, Gmbh, Berlin). The mixture was sonicated for several minutes until an unstable but uniform dispersion was made. The vial was put on the cover of an ultrasonic cleaner and was gently shaken for 1 hour. In this manner, the PMMA spheres self-assembled into a colloidal crystal. Some ethanol was withdrawn and the remaining ethanol was allowed to evaporate for 1 to 2 days. The resulting colloidal crystal arrays were heated at 120° C. for 24 hours to anneal the particles.

(2) Hydrogel Infiltration.

Two polymerization methods were employed to synthesize the hydrogels. For thermo-initiation, 10 μL of 2% potassium peroxodisulfate (K₂S₂O₈) and 0.1 mL of water were added to 0.5 mL of degassed monomer solution (30% w/w Acrylamide (AAm, from Sigma) with various amounts (1 to 10 wt. %) of N,N′-methylene-bisacrylamide (NMBA, from Sigma) as cross-linking agent). The mixture was infiltrated into the PMMA arrays and then polymerized at 70° C. for 12 hours. For redox initiation, 0.5 mL of monomer solution, 0.1 mL of 0.05 M L-ascorbic acid and 10 μL of 2% K₂S₂O₈ were mixed. The mixture was infiltrated into the PMMA array, and polymerization was carried out to completion at room temperature for 12 hours.

The resulting gel was soaked in tetrahydrofuran (THF) for 48 hours to remove the PMMA colloid completely. The ICC scaffold was then soaked in water and allowed to reach an equilibrium swelling state at room temperature.

In addition to this generally known method, improved methodologies for making hydrogel inverted-opal scaffolds were also developed and are described and claimed herein.

In one such improved process for the production of hydrogel inverted-opal scaffolds, approximately 300 scaffolds per batch were made and the testing data indicates that such an improved process may be considered to be a generalized synthesis protocol to produce ICC scaffolds.

The typical process used to make ICC scaffolds depends on utilization of an individual colloidal crystal template within a holder. A severe throughput problem arises with this approach, as each holder makes one scaffold a time. In order to overcome such a limitation and thereby enable much greater throughput, a process for large scale production of ICC scaffolds, through the use of tube holders, has been developed. This process can be used to make a long cylindrical master scaffold (see e.g., FIG. A). Each cylindrical master scaffold, in turn, can be cut into many individual scaffolds. By using 10 glass tubes of 5 mm diameter×3cm high, 10 master scaffolds can be made per batch. Each master scaffold can be cut into about 25-30 round-disk scaffolds of 5 mm diameter×1 mm thick. Therefore, 250-300 scaffolds per batch can be produced. Indeed, additional scaffolds (i.e., increased capacity) per batch can be produced simply by using additional glass tubes. The inverted-opal structures of individual scaffolds cut from the cylindrical master are indistinguishable from those made via a single mold procedure. Using such a methodology, a variety of hydrogel scaffolds have been prepared and examined, including PAAM (polyacrylamide), PHEMA (poly 2-hydroxyethyl methacrylate), pHEA(poly 2-hydroxyethyl acrylate), composite scaffolds (including copolymerized hydrogel), inorganic nanoparticle (TiO₂ and Ca₃(PO₄)₂ doped scaffolds, and positively charged scaffolds.

Previously, investigators have used the thermo-initiation and redox-initiation methods to prepare PAAM (polyacrylamide) scaffolds. Both of these methods require at least 12 hours for the formation of a hydrogel. In order to accelerate the polymerization of hydrogel and enable the large-scale production of hydrogel scaffolds, a rapid photo-polymerization technique to prepare PAAM from the corresponding monomer was developed and is disclosed and claimed herein. The procedure is as follows: 0.4 ml 5% NMBA (N-N′-methylene-bisacrylamide) solution was added to 1 ml 30% acrylamide to form a uniform mixture solution. Approximately 0.001 g IRGACURE (IRG) 2959 (from Ciba Specialty Chemicals, http://www.cibasc.com) powder was added to the mixture solution, which was used for two experiments. (1) 0.5 ml of the solution was polymerized under 365 nm UV light for 30 min. PAAM gel with good strength was obtained. (2) The mixture was infiltrated into the non-uniform sized PMMA arrays and then polymerized under 365 nm UV light for 30 min. The resulting gel with PMMA spheres was soaked in THF to remove the PMMA microspheres for 3 days and then was soaked in water for 1 hour. The inverted-opal polymer PAAM scaffold was obtained. The PAAM scaffold has good inter-cavity connection channels despite the non uniform size of the pores, as observed by confocal microscopy. The photopolymerization technique also works with monodisperse PMMA arrays to make ICC scaffolds.

By definition, composite materials aggregate some of the advantages of every component therein. pHEMA hydrogel, for example, has free —OH groups and PAAM hydrogel, as another example, has free —NH₂ groups. Both —OH and —NH₂ groups are hydrophilic and are, therefore, beneficial to cell growth. It holds, therefore, that cells will grow better in a composite scaffold than in a single-composition scaffold. pHEMA-pAAM composite scaffolds were prepared and tested with respect to cell growth. The procedure for the production of PHEMA-PAAM composite scaffolds is as follows: 4 mL of HEMA monomer, 2 mL of water, 4 drops of ethyl glycol dimethacrylate, and approximately 1 mg of Irgacure (IRG) 819 were mixed to form solution I. 1 ml 30% AAM (acrylamide) solution, 0.4 ml 5% NMBA solution, 2 ml solution I, and 1 mg IRG 2959 were mixed to obtain solution II, which was infiltrated into the PMMA arrays and then polymerized under 365 nm UV light for 30 min. The resulting gel with spheres was soaked in THF to remove the PMMA microspheres for 3 days and then was soaked in water for 10 min. The PHEMA-PAAM composite scaffolds were obtained.

Four types of organic-inorganic mixtures were prepared in order to make composite scaffolds. The constituents of each of the four types of organic-inorganic mixtures are given in Table 1.

TABLE 1
The components of mixtures I-IV

| | | | | |
|---|---|---|---|---|
| Mixture | 0.05 ml 5% | 2 ml 30% | 0.8 ml 5% | 1 mg |
| I | TiO₂ | AAM | NMBA | IRG819 |
| | nanoparticle | | | |
| | (NP) | | | |
| | suspension | | | |
| Mixture | 0.05 ml 5% | 4 ml HEMA + | 4D EGDA | 1 mg |
| II | TiO₂NP | 2 ml H₂O | | IRG 819 |
| | suspension | | | |
| Mixture | 0.05 ml 5% TiO₂ | 4 ml HEA + | 2D EGDA | 1 mg |
| III | NP suspension | 2 ml H₂O | | IRG 819 |
| Mixture | 0.05 ml | 4 ml HEMA + | 4D EGDA | 1 mg |
| IV | Ca₃(PO₄)₂ NP | 2 ml H₂O | | IRG 819 |

Where EGDA is ethylene glycol dimethacrylate.

Each monomer mixture was infiltrated into a PMMA colloidal crystal template. The composite of beads and solution was polymerized under 365 nm UV light for 1 hr for mixture I, II, and IV, and 2 hr for mixture III. The resulting gel with beads was soaked in THF to remove the PMMA template for 3 days and then was soaked in water for 10 min. The corresponding scaffolds were obtained. Every rod-like scaffold can be cut into about 20 round disk-shaped scaffolds of 5 mm diameter by 2 mm thick. The microstructures of the PMMA-TiO₂ scaffolds, PHEMA-TiO₂ scaffolds, pHEMA-Ca₃(PO₄)₂ scaffolds, and PHEA-TiO₂ scaffolds show a regular, porous structure with good inter-cavity connection channels. The pHEA-TiO₂ scaffolds are more transparent than pAAM-TiO₂, pHEMA-TiO₂, and pHEMA-Ca₃(PO₄)₂.

The production of composite scaffolds demonstrates the ability to incorporate another material into the hydrogel scaffold. Nutrients, growth factors, differentiation factors, protein, DNA, peptides, cytokines, chemokines, and drugs could also be incorporated into the scaffold during production. Care must be taken to choose a scaffold preparation method that does not destroy or damage the compound; for example, high temperatures must be avoided as demonstrated above by using UV initiation instead of thermo-initiation.

Positively charged pHEMA+ scaffolds are made from a co-polymer of pHEMA [(poly(2-hydroxyethyl methacrylate)] and pMAETAC [poly(2-(Methacryloxy) ethyl] trimethylammonium chloride )]. The ammonium group is positively charged. The preparation of positively-charged scaffolds is as follows: 0.5 ml of HEMA mixture (1:4 volume ratio of HEMA/H₂O, 1 drop of EGDA) and 50 μl of MAETAC was mixed. The mixture was infiltrated into the polystyrene bead templete and put under 365 nm UV light for photopolymerization for 1 hr and then put into THF solution for 3 days to remove polystyrene and to obtain the pHEMA-pMEATAC scaffold.

### EXAMPLE 2

### Nutrient Release Controlled by Scaffold Permeability

I. Method of Testing Diffusion-In and Release

Nutrient carrier materials are capable of holding and releasing nutrients. For different drug delivery and cell culture purposes, however, the nutrient releasing behaviors such as releasing time and rate, need to be variable and better controlled. In order to increase the variability and control of such behaviors, the cross-linking degree of the scaffold was varied and investigated. The 3D inverted colloidal crystal hydrogel scaffolds, denoted as S1 (round shape: 0.55 cm in diameter and 0.25 cm in height) and S2 (rectangle shape: 1 cm×0.5 cm×0.5 cm), used for the nutrient delivery investigation were made by infiltrating polymers within the voids of hexagonal colloidal crystal PMMA templates and removing the templates subsequently. A small protein, Alexa Fluor 488-labeled Trypsin Inhibitor from Soybean (SBTI, from Molecular Probes, 21KD, PI 4.5) was selected to simulate a nutrient due to its comparable molecular weight to a nutrient and due to its fluorescence emission intensity being proportional to its concentration. Although only one type of molecule is used in these examples, solutions required for cell growth could incorporate multiple nutrients, growth factors, differentiation factors, protein, DNA, peptides, cytokines, chemokines, and drugs. The SBTI solution used for the experiments is made by dissolving an appropriate amount of SBTI into phosphate-buffered saline (PBS) solution to a final concentration of 40 ng/mL. This is double the concentration level (5-20 ng/ml) of the cytokines used for T and B cell production from precursor cells. Such a concentration was selected as a balance between good fluorescence signal and the concentration actually used for differentiation.

The incorporation of the nutrient (i.e., SBTI) into the hydrogel scaffolds was conducted by introducing hydrogel scaffolds into 4ml SBTI solution (40 ng/mL) for nutrient diffusion-in. The method for the diffusion-in testing is to measure the fluorescence intensity change of SBTI PBS solution with the stand-in time of hydrogel scaffolds. As the fluorescence intensity of the SBTI PBS solution is proportional to SBTI concentration, once the SBTI molecules diffuse into the hydrogel scaffolds, the SBTI concentration in solution decreases, thereby resulting in the decrease of the fluorescence intensity of the SBTI PBS solution as well.

After the hydrogel scaffold is saturated with SBTI, the release of the SBTI out of the hydrogel scaffold was tested by bringing the SBTI saturated scaffolds into contact with 4 mL fresh PBS solution. Due to the release of SBTI out of the hydrogel scaffolds, the SBTI concentration in PBS solution increases, increasing the fluorescence intensity. Therefore, by monitoring the fluorescence intensity variation of the solution, the kinetics for SBTI diffusing into and release out of hydrogel scaffolds can be measured. Additionally, the relationship between the fluorescence intensity of SBTI solution and its concentration can be calibrated. For example, it is found that the fluorescence intensity of the SBTI solution is linearly related to its concentration. It is possible, therefore, to quantitatively calculate the amount of nutrient (i.e., the SBTI) that diffuses into and releases out of the hydrogel scaffolds by the change in the fluorescence intensity.

II. Diffusion-In Kinetics.

The fluorescence labeled SBTI PBS solution has a strong emission peak centered at 516 nm under the excitation wavelength of 495 nm. The fluorescence intensity of the SBTI PBS solution is largely dependent on the concentration of SBTI in the solution—i.e., the relationship is linear. As the concentration of SBTI decreases, the fluorescence intensity of the solution decreases accordingly. When the hydrogel scaffolds are introduced into the SBTI PBS solution, the SBTI molecules diffuse into the porous structures of the hydrogel scaffolds, and thereby the concentration of SBTI in solution decreases and, as a consequence, the fluorescence intensity of the solution decreases. As the stand in time of the scaffolds is increased, a greater amount of SBTI diffuses into the scaffolds, and the fluorescence intensity of the solution will continue to decrease. When the scaffolds are saturated with SBTI, however, the diffusion reaches an equilibrium point and the fluorescence intensity becomes stable (i.e., no further decrease or increase in fluoresecence is observed). The kinetics of the nutrient as it diffuses into the hydrogel scaffolds can thus be measured.

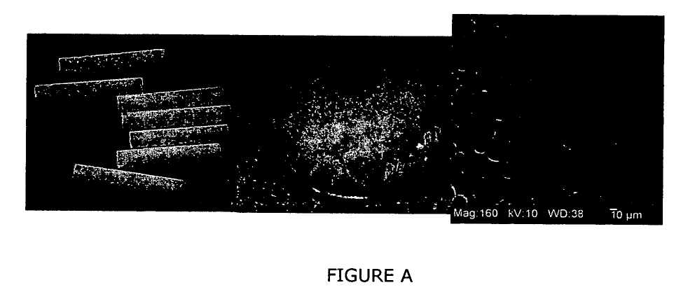

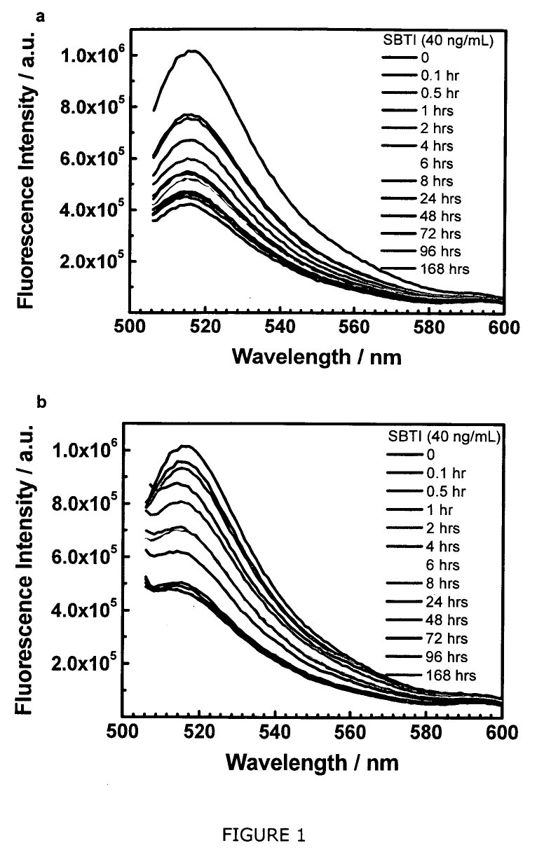

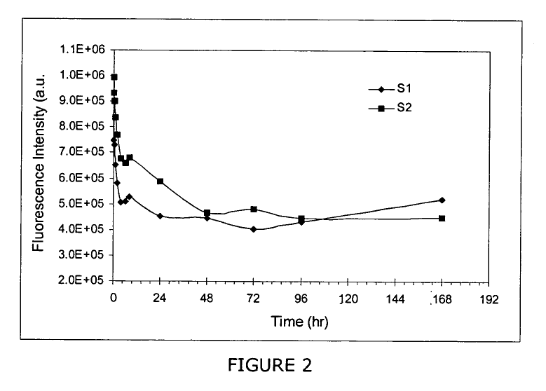

FIG. 1 charts the kinetics of SBTI as it diffuses into the hydrogel scaffolds. The hydrogel scaffolds of S1 and S2 were tested under identical conditions. The two scaffolds were prepared according to different processes, however, and each scaffold has a different permeability property due to the differing degree of polymer cross-linking. The S2 scaffold is “harder”—i.e., it has a higher degree of cross-linking, while the S1 scaffold is “softer” and has a very ordered porous structure and features a nearly transparent appearance. The fluorescence intensity changes of the scaffolds were monitored for 168 hours (i.e., 7 days). The curve for SBTI (40 mg/mL) in FIG. 1 represents the fluorescence of SBTI PBS solution at a concentration of 40 ng/mL prior to the introduction of hydrogel scaffolds. FIG. 1 shows both SBTI PBS solutions after the scaffolds were first introduced and that the fluorescence intensities decrease quickly in the first three days and then slow and finally stabilize after 3 days, as indicated by the fluorescence intensity variation. Due to the different properties of scaffolds, however, the fluorescence intensity decreases differently for each scaffold—i.e., the decrease is much faster for S1, thereby indicating that there are different diffusion kinetics and absorption abilities for each of the scaffolds. The kinetics curves shown in FIG. 2 indicate that the decrease in fluorescence intensity for each of the scaffolds follows an exponential decay and decreases more for S1 in comparison to S2 within the same time span, suggesting that the S1 scaffold has a stronger nutrient absorbing ability than the S2 scaffold. This is most likely due to the relatively low level of cross linking in the S1 scaffold, which allows the molecules to more easily penetrate into the pores of scaffolds.

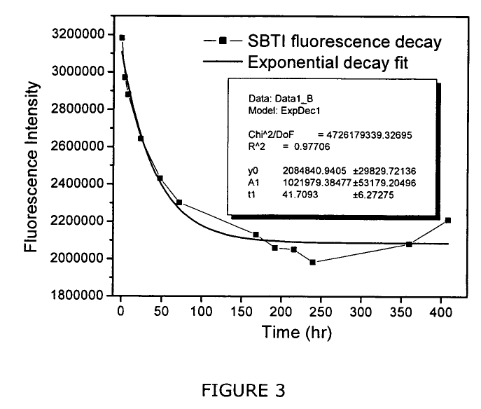

The fluorescently labeled SBTI PBS solution itself has an inherent decrease in fluorescence intensity. FIG. 3 indicates the fluorescence decay of SBTI in solution over a period of time. The results indicate that the fluorescence of a fluorescence-labeled SBTI solution exponentially decays over time, although, the fluorescence intensity appears to remain constant after 7 days. Such a decay could be due to any one of a myriad of reasons—e.g., photobleaching, decomposition of the fluorescent dye, concentration quenching due to the aggregation of the protein over time, etc. The actual fluorescence intensity decrease due to the diffusion of SBTI into the scaffolds, therefore, can be obtained by subtracting the fluorescence intensity decrease of SBTI itself from the total decay of the SBTI solution.

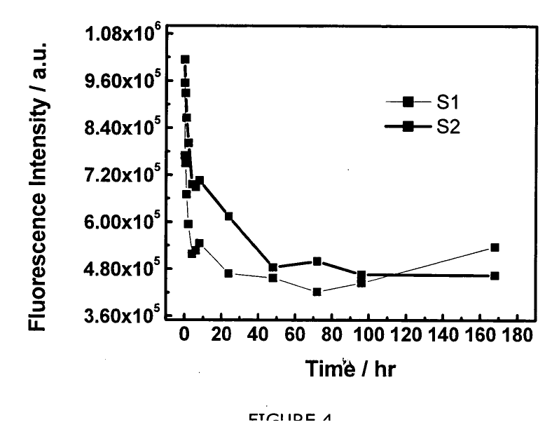

FIG. 4 represents the real fluorescence intensity change due to SBTI diffusion into the hydrogel scaffolds—i.e., it reflects the actual kinetics of SBTI diffusion into the hydrogel scaffolds. It is shown that the actual time for SBTI diffusion into the hydrogel scaffolds is around 24 hours, with most of the diffusion occurring within the first four hours. Such a result indicates that the use of hydrogel scaffolds to store nutrients is highly advantageous.

In the above discussion, diffusion into the scaffold occurred under passive conditions. The flow of nutrients into the scaffold could also be increased and controlled by active transport, such as placing the scaffold in a flow cell and flowing nutrient solution through the scaffold. An alternative active transport method includes pressure infiltration such as injecting nutrients into the scaffold using a syringe needle.

III. Release Out Kinetics.

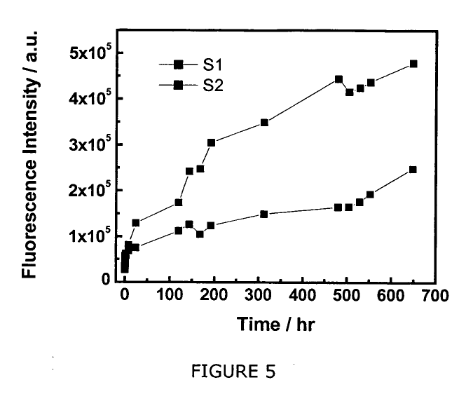

The kinetics of the diffusion-out of SBTI from the scaffolds were measured by placing the SBTI saturated scaffold in fresh PBS solution. FIG. 5 shows the release kinetics of SBTI out of S1 and S2 hydrogel scaffolds. The data in FIG. 5 indicates that the SBTI is released out of hydrogel scaffolds over a period of time of 20 days, which is a typical and appropriate time period for nutrients to be delivered to T and B cell cultures. The slow release of nutrient out of the scaffolds makes them ideal matrices for cell culture—i.e., not only are they a solid matrix but they are also capable of being a nutrient supplier. The amount of SBTI released out, however, is different for scaffolds of differing composition. In comparison with the S2 scaffold, the S1 scaffold releases more SBTI, suggesting that the lesser degree of cross-linking in the polymer results in an easier or quicker release of nutrient—a corollary to the S1's similar nutrient diffusion-in properties. From these results, it can be concluded that the properties of the hydrogel scaffolds (e.g., cross-linking degree, pore size and elasticity) play an important role in controlling the nutrient diffusion-in and out of the scaffold. It is thus possible to manually control such parameters during the scaffold making process in order to optimize such properties for controlled nutrient delivery.

IV. Inorganic and Hydrogel Scaffolds with LBL Coatings

The controlled release behavior of nutrients from inorganic and hydrogel scaffolds were also compared. Scaffolds were made from Na₂SiO₃ (inorganic) and pHEMA (hydrogel) materials, respectively, and were thereafter subjected to nutrient release experiments. The 3D ordered inorganic and hydrogel scaffolds differ in hardness, pore size and elasticity which has a significant influence on nutrient release properties. In these nutrient release experiments, the SBTI was incorporated into the scaffold using a layer-by-layer (LBL) process. This consists of alternately coating SBTI and its counterpart, poly (diallyldimethylammonium chloride) (PDDA), onto the scaffolds. The coating process is as follows: the scaffolds were first introduced into a 0.5% PDDA solution (pH=4) for 10 min, followed by a rinsing with deionized water, then the PDDA coated scaffolds were brought in contact with the SBTI solution (40 ng/mL) for 10 min, followed again by a rinsing with deionized water, and dried with a stream of nitrogen.

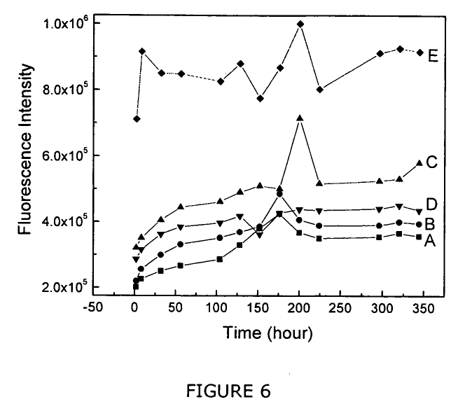

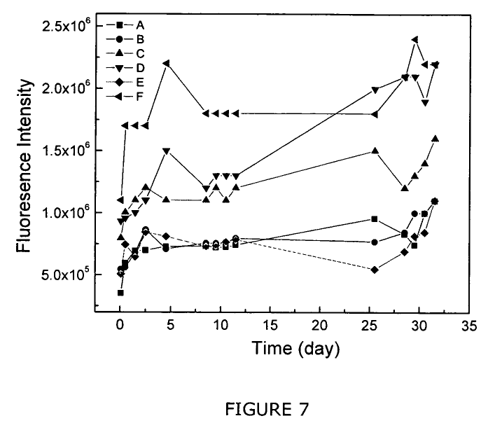

This coating process resulted in the scaffolds being coated with alternating layers of PDDA and SBTI. The process can be repeated any number of times in order to get the desired number of layers and is well within the skill of one of ordinary skill in the art given the present specification. The scaffolds were coated with various bilayers of PDDA and SBTI to produce samples of Na₂SiO₃ scaffold-(PDDA-SBTI)2, Na₂SiO₃ scaffold-(PDDA-SBTI)4, Na₂SiO₃ scaffold-(PDDA-SBTI)6, Na₂SiO₃ scaffold-(PDDA-SBTI)8-PDDA, and Na₂SiO₃ scaffold-(PDDA-SBTI)10 samples, as denoted by A, B, C, D, and E respectively in FIG. 6, and of pHEMA scaffold (PDDA-SBTI)1, pHEMA scaffold (PDDA-SBTI)2, pHEMA scaffold (PDDA-SBTI)4, PHEMA scaffold (PDDA-SBTI)6, PHEMA scaffold (PDDA-SBTI)8-PDDA, and pHEMA scaffold (PDDA-SBTI)10, referred to as A, B, C, D, E and F in FIG. 7, for nutrient release testing.

The coated scaffolds were then separately transferred into 10 mL deionized water for monitoring of the release of nutrient out of the scaffolds—such monitoring occurred by measuring the change in fluorescence intensity. FIG. 6 shows the release kinetics of SBTI out of the LBL coated Na₂SiO₃ scaffolds. It can be observed that the SBTI was slowly released from the scaffolds, as indicated by the gradual increase of fluorescence intensity, and such a release continues for about 10 days. The rate of nutrient release generally increases with increased layers of LBL coating, indicated by increasing fluorescence intensity. The only exception is scaffold D, which gives a lower release rate than C, and this is most likely due to the outermost layer being negatively charged PDDA that slows down the release of SBTI. The total quantities of nutrient released from the coated hydrogel scaffolds of A, B, C, D, and E are calculated as 37.5, 41.5, 60.9, 45.7 and 96.3 ng, respectively, as calculated by the relationship between the fluorescence intensity and SBTI concentration. This result indicates that the nutrient release can be modulated by varying the number of coating layers as well as by varying the materials used to coat.

FIG. 7 shows the release kinetics of SBTI out of the LBL coated PHEMA hydrogel scaffolds. The data indicates that the SBTI release out of PHEMA hydrogel scaffolds may last up to 30 days, much longer than the release of SBTI out of the Na₂SiO₃ scaffolds which continue to release SBTI for up to 10 days. The longer time for nutrient release ensures that the persistent nutrient requirements required for cell growth and differentiation over the course of several weeks are met. When PDDA is coated in such a manner that it constitutes the outermost layer, the release of nutrient out of both Na₂SiO₃ and PHEMA scaffolds is slower, and requires a longer period of time, as shown by curves D and E in FIGS. 6 and 7, respectively. The quantities of nutrient released from the coated hydrogel scaffolds of A, B, C, D, E and F are calculated as 115.8, 115.8, 136.8, 231.6, 115.8 and 231.6 ng, respectively, indicating that in comparison with inorganic Na₂SiO₃ scaffold, more nutrients can be incorporated into and released out of PHEMA hydrogel scaffolds, even though the scaffolds have the same coating layers.

### EXAMPLE 3

### Nutrient Release Controlled by Coating Hydrogel Scaffolds

As indicated herein, through natural diffusion, hydrogel scaffolds can be saturated with SBTI within 4 hrs. The release of SBTI out of the scaffolds, however, can last up to 27 days. For special drug delivery or cell culture purposes, the time of release of nutrient out of the scaffold needs to be variable. For this purpose, a method using LBL assembly was developed to coat polyelectrolytes onto hydrogel scaffolds that have been previously saturated with nutrient in order to control the releasing properties of the scaffold. Coating polyelectrolytes onto the hydrogel scaffolds after the scaffold is saturated prevents a fast release of the nutrient from the scaffolds. In fact, results demonstrate that LBL coating layers decrease the releasing rate of SBTI from the saturated hydrogel scaffolds.

The nutrient release behaviors of hydrogel scaffolds without any coating layers and with a five bilayer coating of polystyrene sulfonate (PSS) and polyethylimine (PEI) were compared. The scaffolds were first saturated with nutrient by the natural diffusion process as described herein. After the scaffolds were saturated with SBTI, one scaffold was coated immediately with five bilayers of PSS and PEI by alternatively dipping the scaffold into PSS and PEI solutions (both at 1 mg/mL), while the other scaffold was not modified. The two scaffolds were then introduced into fresh PBS solutions (4 mL) for the nutrient release study.

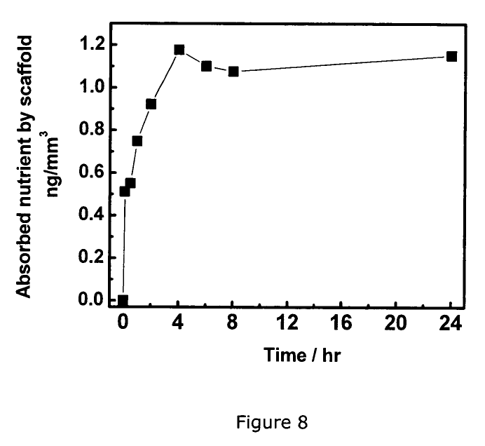

FIG. 8 shows the amounts of SBTI that diffused into the hydrogel scaffolds during the initial loading and saturation. By calculating the SBTI concentration from the decrease in fluorescence intensity, the diffusion quantity of SBTI into the scaffolds was determined. As indicated, the scaffold absorbed 1.08 ng/mm³ of SBTI within 4 hours, as shown in FIG. 8.

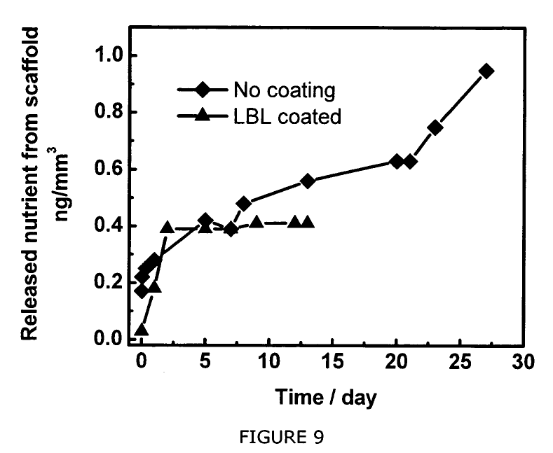

The release of the absorbed SBTI from the scaffolds with and without LBL coating is quite different, as shown in FIG. 9. For the release of SBTI from the scaffold without LBL coating, 88% of the previously absorbed nutrient (0.95 of 1.08 ng/mm³) could be released, and the releasing process lasts 27 days. For scaffolds with LBL coating, only 39% of the previously absorbed nutrient (0.42 of 1.08 ng/mm³) is released out, and the release time was 2 days. The difference in release kinetics between the two types of scaffold is attributed to the coating layers. Thus, the precise control of nutrient release out of hydrogel scaffolds can be achieved by coating the scaffolds with differing amounts of polyelectrolyte layers and different materials in the polyelectrolyte layers.

Before releasing, the nutrient needs to be incorporated into the scaffold. In summary and from the data shown in Examples 1-3, it would be apparent to one of ordinary skill in the art that there are numerous ways to incorporate the nutrient into the scaffold and would be known to a skilled artisan given the present specification. For sake of explanation, but not by way of limitation, two such methods are outlined herein. First, the nutrient and its counterpart can be incorporated onto the scaffold by LBL coating. This results in the loading of a large amount of nutrient onto the scaffolds. Second, the nutrient can be diffused into the scaffolds by placing the scaffolds into different concentrations of nutrient solution. The LBL coating method may incorporate more nutrient into the scaffolds, while the natural diffusion method may incorporate less. The release of nutrient out of scaffolds is more controlled where the nutrient had been incorporated into the scaffold by natural diffusion. This is shown by the results outlined in examples 2 and 3, herein. According to requirements and uses by the user, different nutrient incorporation methods can be employed to incorporate a desired amount of nutrient into the scaffolds. The controlled release of the nutrient after incorporation can also be achieved.

### EXAMPLE 4

### Release Kinetics of Interleukin-3

The short term and long term release kinetics of Interleukin-3 (IL-3) from PAAM scaffold were investigated through the use of Enzyme-Linked Immunosorbent Assay (ELISA) to measure the amount of IL-3 released.

PAAM scaffold was immersed in 1 ml RPMI-1640 Medium (RPMI-1640 was developed by Moore et. al. at Roswell Park Memorial Institute, hence the acronym RPMI) with 10% fetal bovine serum (FBS) and 50 ng/mL IL3 for 24 hours. Scaffold was then placed in the well of a 48 well plate containing 500 uL of RPMI media with 10% FBS but no IL-3 and incubated at 37° C., 10% CO₂. For the short term release study, the media in the well was collected and replaced with fresh media at different time-points—i.e., 30 seconds, 1 minute, 5 minutes, 1 hour, 5 hours, 7 hours, 22 hours. For the long term release study, the media in the well was collected and replaced with fresh media every day for 6 days. Collected media were transferred to a different well of the same plate. The amount of IL-3 in the media collected was estimated using a commercial ELISA kit.

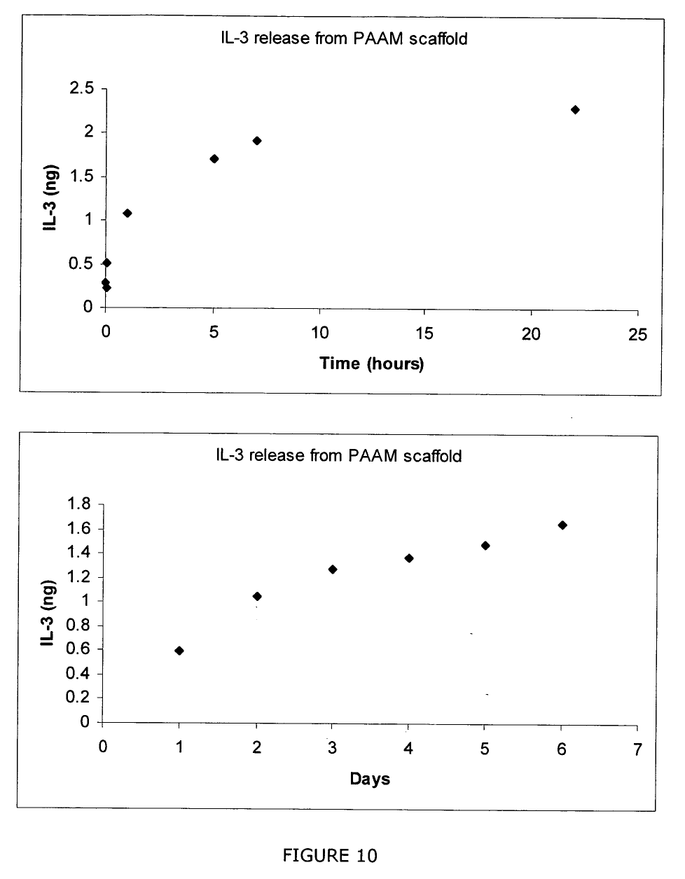

The results from these experiments (FIG. 10) indicate that PAAM scaffolds can be successfully loaded with IL-3 and are capable of slow release of the cytokine. The apparent variation in the amount of cytokine released between the first and second experiments is most likely due to the denaturing of the IL-3 following prolonged storage in the incubator at 37° C. or may be possibly due to the structural variation between individual scaffolds or a combined effect of these.

### EXAMPLE 5

### Loading and Controlled Release of Interleukin 3 from Hydrogel that promotes Proliferation of TF-1 Cells

Human bone marrow erythroblast cells TF-1 (from ATCC, #CRL-2003.) respond to a variety of lymphokines and cytokines including IL-3. The proliferation of the cell line depends on the presence of the IL-3 or granulocyte-macrophage colony-stimulating factor (GM-CSF). These cells were used as a model system in order to show that the controlled release of cytokines from a scaffold does indeed provide nutrient and growth/differentiation factors to the cells in the culture.

The TF-1 cells obtained from the ATCC were expanded in a T-25 flask using ATCC recommended medium: RPMI 1640 medium with 2 mM L-glutamine adjusted to contain 1.5 g/L sodium bicarbonate, 4.5 g/L glucose, 10 mM HEPES, 1.0 mM sodium pyruvate and supplemented with 5 ng/ml IL-3 and 10% fetal bovine serum. In some experiments, as noted below, IL-3 was not used or the concentration of IL-3 was changed, as noted.

Four hydrogel scaffolds of the size 4×4×4 mm³ were sterilized in 70% isopropyl alcohol (IPA) overnight, and then washed with sterile water twice. Two of the cleaned scaffolds were then put into the ATCC recommended TF-1 growth medium with 50 ng/ml IL-3. Two additional cleaned scaffolds were put into an IL-3 free medium. All were incubated at 37° C. overnight. Finally, the scaffold was removed from the media and washed with IL-3 free media briefly before use. 50 ng/ml IL-3 was chosen for loading and the final concentration of IL-3 released was expected to be equivalent to 5 ng/ml, the normal culture condition.

The TF-1 cells were harvested from a flask and centrifuged at 136 G (800 rpm) for 5 minutes in order to remove the medium, and re-suspended in IL-3 free medium. TF-1 cells were seeded into a 48 well, non tissue culture treated sterile plate at a final concentration of 2×10⁵ cells/ml, 0.5 ml/well. Two wells had one IL-3 loaded scaffold each, two wells had one scaffold without IL-3, two wells had media without IL-3 and two wells had media with 5 ng/ml IL-3. The plate was cultured in a 37° C. and 5% CO₂ incubator for 3 days, and the cells in the media of each well were thereafter counted using a hemacytomter.

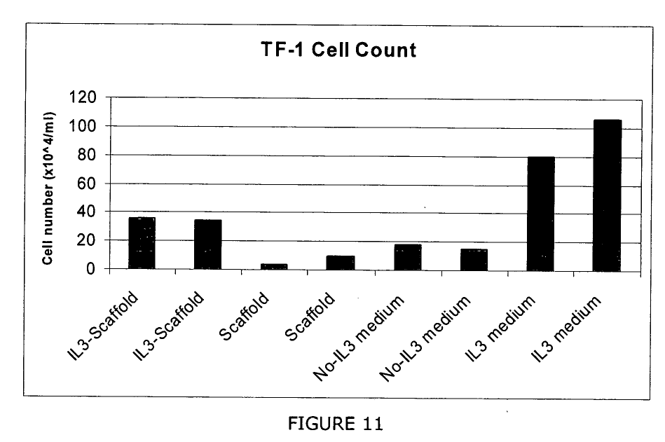

The results are shown in FIG. 11. As can be clearly seen, the number of cells in the wells containing IL-3 loaded scaffold was lower than the number of cells in the wells with IL-3 medium (no scaffold). However, the number of cells in the wells with IL-3 loaded scaffold were still significantly higher (5-9 times) than the number of cells in the wells without IL-3 (either with or without scaffold). These results about the number of cells were also confirmed with an MTT cell proliferation assay.

In addition, confocal microscope images revealed that more cells were found to be trapped in the IL3 loaded scaffolds than were trapped in the unloaded scaffold (with IL3 present in the media). There were very few cells present in the scaffold for the sample without IL3 (in either the media or the scaffold). This suggests that the reason for the difference in the number of cells found in the media of the IL3 loaded scaffold sample and in the media of the IL3 in media sample could be that although a similar number of cells proliferated, the cells were trapped in the IL3 loaded scaffold rather than being in the media. The cells may be attracted to the scaffold due to the high local concentration of IL3.

### EXAMPLE 6

### IL-3 Release from Composite Scaffolds

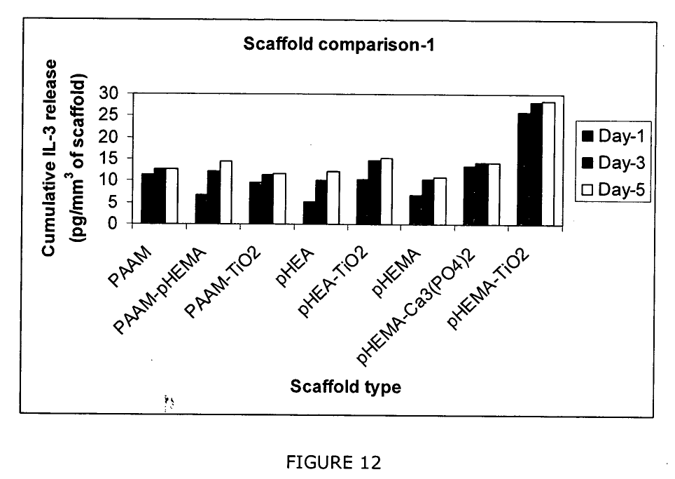

We compared IL-3 controlled release from different formulations of hydrogel and composite ICC scaffolds. IL-3 release from eight different scaffold materials (viz. PAAM, pAAM-pHEMA, pAAM-TiO₂, PHEA, pHEA-TiO₂, pHEMA, pHEMa-Ca₃(PO₄)₂, and pHEMA-TiO₂) was studied. Scaffolds were immersed overnight in media containing 50 ng/ml IL-3. The scaffolds were then transferred into fresh media without IL-3. Media was collected and replaced with fresh media on days 1, 3 and 5. The collected media was assayed (ELISA) to determine the level of IL-3 released from each scaffold (FIG. 12).

There was no major difference in IL-3 release among the different scaffolds. Doping with different inorganic materials like Ca₃(PO₄)₂ and TiO₂ nanoparticles was attempted to increase the mechanical strength of these composite scaffolds. These treatments did not result in any major change in IL-3 release from any of the scaffolds tested except for PHEMA. pHEMA-TiO₂ showed an almost 2.5× increase in IL-3 release compared to untreated PHEMA.

### EXAMPLE 7

### IL-3 Release and Cell Growth with Positively Charged Scaffolds

With respect to the positively charged scaffold PHEMA+, the diameter changes from 0.35 to 1.2 cm and the thickness changes from 0.1 to 0.4 cm, when the scaffold is put into pure water. This is a reversible change and the size is similar to other scaffolds when this scaffold is put into culture media or water with ions such as Na₂SO₄. This swelling ability is most likely due to the repulsion between the positively charged ammonium groups inside the polymer. Anions in the solution can neutralize the charge and thus reduce the size of scaffold.

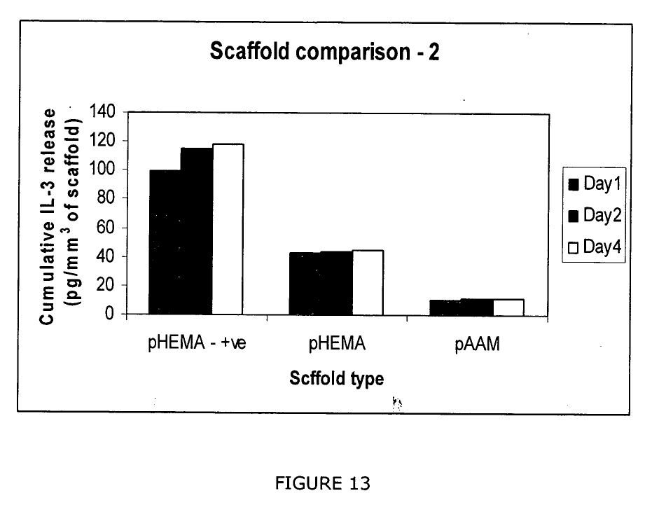

The swelling property of the positively charged scaffold may also be used for the incorporation of nutrients or cytokines into the scaffold. Hydrogels which swell and/or shrink and/or change permeability/porosity in response to temperature or pH changes are also known and could be used to make inverted-opal scaffolds. A positively charged scaffold was tested with IL-3 loading and release. The results of such tests show that the positively charged scaffold had improved IL-3 release compared to the other hydrogel scaffolds (FIG. 13). In this experiment, PHEMA+, pHEMA and PAAM scaffolds were incubated with media containing 50 ng/mL of IL-3. Incubation time was doubled to 48 hrs at 37° C. The scaffolds were then washed with IL-3 free media and replaced in fresh, IL-3 free, media in wells of a 96 well plate. Media was collected and replaced on days 1, 2 and 4. The collected media was assayed (ELISA) in order to determine the level of IL-3 released from the different scaffolds.

A sustained release of IL-3 was observed from positively charged PHEMA on all days tested. The amount of IL-3 released per unit volume of the scaffold was 2× more compared with pHEMA and 9× more compared to PAAM. Loading of the scaffold with IL-3 for a longer duration increased the loading of PHEMA scaffold but produced no change in PAAM loading.

The proliferation of human bone marrow erythroblast cell TF-1 depends on the presence of IL-3. TF-1 cells were cultured in 48 well microplates at 3 different conditions:
(1) in the presence of a ICC scaffold loaded with IL-3;
(2) in the presence of the same ICC scaffold without IL-3 loaded, but with IL-3 supplied in the medium;
(3) IL-3 supplied in either media or scaffold.

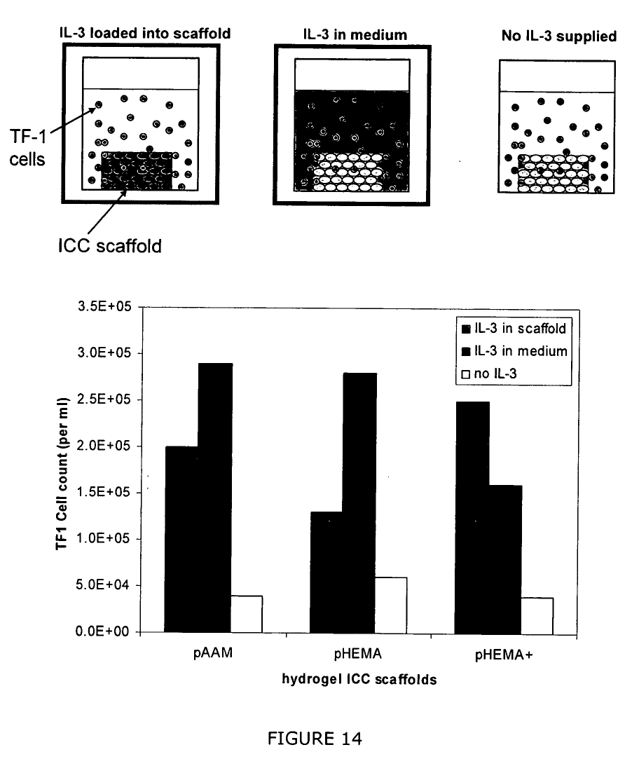

pAAM, PHEMA and positively charged PHEMA+ ICC scaffolds were each tested under these conditions. The results indicate that the IL-3 released from the hydrogel scaffold can support TF-1 growth similar to IL-3 presented in the medium (FIG. 14), and again, positively charged scaffolds showed better cell proliferation.

These results clearly demonstrated that hydrogel ICC scaffold can release growth factors continuously, and regulate cell proliferation and/or differentiation in a controlled fashion. In addition, biodegradable hydrogel materials, such as poly(lactic-co-glycolic acid), can also be used to make hydrogel inverted opal scaffold and be able to provide controlled release of incorporated nutrients by means of the degradation induced release process.

### REFERENCES

1. N. A. Kotov and S. Wang, U.S. patent application Ser. No. 10/460,059 “3D Tissue Constructs on the basis of colloidal crystals surface modified by sequential layering”
2. Y. Zhang, S. Wang, M. Eghtedari, M. Motamedi, N. A. Kotov, Inverted Colloidal Crystal Hydrogel Matrices as Three-Dimensional (3D) Cell Scaffolds, Advanced Functional Materials, 2005, 15, 725-731.
3. N. A. Kotov, Y. Liu, S. Wang, C. Cumming, M. Eghtedari, G. Vargas, M. Motamedi, J. Nichols, J. Cortiella, Inverted Colloidal Crystals as 3D Cell Scaffolds, Langmuir, 2004, V 20, No.19, 7887-7892.
4. Y. Liu, S. Wang, J. W. Lee, N. A. Kotov, A Floating Self-Assembly Route to Colloidal Crystal Templates for 3D Cell Scaffolds, Chem. Mater., 2005 17(20); 4918-4924.
5. D. J. Irvine, A. Stachowiak, S. Jain, Engineered Biomaterials for Control of Immune Cell Functions, Materials Science Forum, 2003, 426, 3213-3218.
6. S. Shanbhag, S. Wang, N. A. Kotov, Cell Distribution Profiles in Three-Dimensional Scaffolds with Inverted Colloidal Crystal Geometry: Modeling and Experimental Investigations, Small, 2005, V 1, No. 12, 1208-1214.
7. S. Shanbhag, J. W. Lee, N. A. Kotov, Diffusion in three-dimensionally ordered scaffolds with inverted, colloidal crystal geometry, Biomaterials 26 (2005) 5581-5585.

1. A method for the controlled release of one or more constituent compounds, comprising the steps of:
providing an inverted-opal scaffold, wherein the inverted-opal scaffold comprises a hydrogel material; and
incorporating one or more constituent compounds in the inverted-opal hydrogel scaffold, wherein the one or more constituent compounds incorporated in the inverted-opal hydrogel scaffold are capable of being released from the inverted-opal hydrogel scaffold in a controlled manner.

2. The method of claim 1, wherein the constituent compounds are selected from the group consisting of biologicals, chemicals, polymers, naturally occurring compounds, synthetic compounds, and combinations thereof.

3. The method of claim 1, wherein the constituent compounds are selected from the group consisting of nutrients, growth factors, differentiation factors, protein, DNA, peptides, cytokines, chemokines, drugs, and combinations thereof.

4. The method of claim 1, wherein the incorporation of the one or more constituent compounds into the inverted-opal hydrogel scaffold occurs by mixing the one or more constituent compounds with the precursor materials used to produce the inverted-opal hydrogel scaffold.

5. The method of claim 1, wherein the incorporation of the one or more constituent compounds into the inverted-opal hydrogel scaffold occurs by diffusion into the inverted-opal hydrogel scaffold after its production.

6. The method of claim 1, wherein the incorporation of the one or more constituent compounds into the inverted-opal hydrogel scaffold occurs by active transport into the inverted-opal hydrogel scaffold after its production.

7. The method of claim 1, wherein the kinetics of the controlled release of the one or more constituent compounds are controlled by the concentration of constituent compounds incorporated into the inverted-opal hydrogel scaffold.

8. The method of claim 1, wherein the kinetics of the controlled release of the one or more constituent compounds are controlled by controlling one or more properties of the inverted-opal hydrogel scaffold, wherein the one or more properties controlled are selected from the group consisting of permeability, pore size, channel size, degree of cross-linking, elasticity, hardness, and combinations thereof.

9. The method of claim 1, wherein the kinetics of the controlled release of the one or more constituent compounds is controlled by further including a coating onto the surface of the inverted-opal hydrogel scaffold.

10. The method of claim 9, wherein the coating is coated onto the surface of the inverted-opal hydrogel scaffold by a layer-by-layer methodology.

11. The method of claim 10, wherein the kinetics of the controlled release of the one or more constituent compounds is controlled by the number of layers or materials used in the coating.

12. The method of claim 1, wherein the kinetics of the incorporation or the controlled release of the one or more constituent compounds is controlled by one or more environmental parameters selected from the group consisting of salt concentration, temperature, pH, and combinations thereof.

13. The method of claim 1, wherein the kinetics of the controlled release of the one or more constituent compounds is controlled by the degradation of the hydrogel material in the inverted-opal hydrogel scaffold.

14. The method of claim 1, further including at least one secondary material operably associated with the hydrogel that is capable of forming a composite scaffold within the inverted-opal hydrogel scaffold.

15. The method of claim 1, further including at least one secondary material operably associated with at least one of the hydrogel or the inverted-opal hydrogel scaffold, wherein the at least one secondary material is capable of forming a composite scaffold within or with the inverted-opal hydrogel scaffold.

16. The method of claim 1, wherein the inverted-opal hydrogel scaffold has an inverted colloidal crystal structure.

17. A method of providing a 3D microenvironment for cell growth, comprising the steps of:
providing an inverted-opal hydrogel scaffold having a three-dimensional structure;
incorporating one or more constituent compounds into the inverted-opal hydrogel scaffold; and
controlling the release of the one or more constituent compounds into a medium that permeates through the inverted-opal hydrogel scaffold, to thereby provide a predetermined concentration of the one or more constituent compounds for cell growth.

18. The method of claim 17, wherein the predetermined concentration of the one or more constituent compounds for cell growth is an optimal concentration of the one or more constituent compounds capable of optimizing cell growth.
